(19) United States
(12) Patent Application Publication
(10) Pub. No.: US 2005/0078557 A1
(43) Pub. Date: Apr. 14, 2005

(54) Sensor

(76) Inventor: Alf Andersen, Torslanda (SE)

Correspondence Address: HARNESS, DICKEY & PIERCE, P.L.C., P.O. BOX 8910, RESTON, VA, 20195, US

(21) Appl. No.: 10/498,365

(22) PCT Filed: Dec. 12, 2002
(86) PCT No.: PCT/SE02/02313

(30) Foreign Application Priority Data
Dec. 12, 2001 (SE) 0104206-8

(51) Int. Cl. G04F 13/00; G01N 33/00; G01N 33/02; G01N 33/15; G01N 31/22

(52) U.S. Cl. 368/11; 73/73; 116/206; 73/29.04; 73/29.05

(57) ABSTRACT

The present invention relates to sensor for indicating a cumulative time period of presence of an ambient relative humidity, there the sensor comprising an indicator and a carrier and the indicator being located at one or more limited portions of the carrier. The carrier allowing migrating of the indicator within the carrier and the indicator causing an extension thereof by migrating from the one or more limited portions when the ambient relative humidity level exceeds a predetermined level. Further, the extension by migrating being detectable by a user and the cumulative time period being related to the extension by migrating and the characteristics of the indicator and the carrier. The present invention also relates to a method for reading the cumulative time period of presence of an ambient relative humidity indicated by a sensor and a method for providing a sensor for indicating a cumulative time period of presence of an ambient relative humidity by providing a carrier with an indicator.

### TECHNICAL FIELD OF THE INVENTION

This invention relates to a sensor for indicating a cumulative time period of presence of an ambient relative humidity. The present invention also relates to a method for reading a cumulative time period of presence of an ambient relative humidity indicated by a sensor and a method for providing a sensor for indicating a cumulative time period of presence of an ambient relative humidity by providing a carrier with an indicator, wherein the indicator is arranged at one or more limited portions of the carrier.

### BACKGROUND OF THE INVENTION

Exposure to moisture can lead to undesired changes to a product during manufacture, storage, handling or transportation. Examples of moisture sensitive goods may be construction materials, food stuff, pharmaceuticals, boats, buildings, houses, vehicles, electronic equipment and so on. There are some known sensors in the prior art for indicating moisture.

U.S. Pat. No. 4,292,916 describes a timer and a storage condition indicator comprising a carrier mixture and a receptive layer. In this invention a sensor shift is mainly triggered by parameters such as temperature, exceeding a set value.

U.S. Pat. No. 4,098,120 describes a sensor comprising at least an absorbent wick. Herein, the indication of humidity is based on a phenomena called wick action and a course of time during which a material is exposed to moisture, is greatly depending on the amount of available moisture.

Further, U.S. Pat. No. 4,793,180 describes an irreversible humidity indicator having a delayed response time including a porous plastic carrier. However, the humidity level measured is the level inside hollow goods using a complicated device shown in the document.

A disadvantage with the sensors in the prior art is that they preferably indicate the exposure as such for predetermined levels of relative humidity.

### SUMMARY OF THE INVENTION

One object of the present invention is to achieve a sensor which is able to indicate whether a product, an item, an object and/or a material has been exposed to a certain amount of humidity preferably during manufacture, packaging, storage, transport and/or handling and to estimate for how long the product, item, object or material has been exposed to a relative humidity that exceeds a predetermined humidity level. The predetermined humidity level is depending on the products, items, objects or materials sensitivity for humidity.

Another object of the invention is to achieve a sensor how is irreversible, impervious and stable.

According to a first aspect of the invention, the above and other objects are achieved with a sensor for indicating a cumulative time period of presence of an ambient relative humidity there the sensor comprises an indicator and a carrier. The indicator is located at one or more limited portions of the carrier. The carrier allows migration of the indicator within the carrier and the indicator causes an extension thereof by migrating from the one or more limited portions, when the ambient relative humidity level exceeds a predetermined level. Further, the extension by migration is detectable by a user and the cumulative time period is related to the extension by migration and the characteristics of the indicator and the carrier. This means that the carrier allows the indicator to migrate within and/or at a surface layer of the carrier when a predetermined humidity level is exceeded and ceases migration of the indicator when the ambient relative humidity level is lowered below said predetermined level and resuming migration when the ambient relative humidity level again exceeds said predetermined level. An advantage of using a sensor according to the invention compared to a sensor with a so called wick action using capillary rising is that it is possible to indicate a cumulative time period of presence of an ambient relative humidity.

In addition, different kind of products, materials and objects are in various degrees sensitive to moisture and according to the present invention it is possibility to design specific sensors, defining different predetermined humidity levels, by using several carriers and/or indicators.

In a preferred embodiment of the present invention the carrier is at least one of a hygroscopic material and a material that has been given hygroscopic properties during preparation or modification. A hygroscopic material is capable of adsorbing and desorbing moisture, respectively, i.e. water vapour and/or water, depending on the ambient relative humidity of the surrounding atmosphere in such way that equilibrium is achieved. This enables manufacture and use of an irreversible sensor.

Another advantage of using a hygroscopic carrier is that the indicator and the carrier is humid at approximately the same time and therefore capillary rising is avoided. Be using the mentioned carrier the indicator is forced to diffuse through the carrier, i.e. resulting in a diffusion controlled or transport controlled process, which is a much more slowly process compared to a capillary rising process. The transport of different indicator substances is here controlled by their abilities to migrate. For instance, water-soluble molecules are utilised requiring dissolution in a solvent to be able to move.

Further, the migration of an indicator according to the invention preferably occur through diffusion of the indicator or by transportation of the indicator molecules in the water phase, i.e. the indicator molecules, such as a dye, is following the water through the sensor. Suitable materials for a carrier can for instance be paper, fabric and non-woven materials. More preferable the carrier consists of at least one of glass, glass fibre, polyester, polypropylen, cellulose, polymer etc.

In a preferred embodiment of the invention the hygroscopic material is at least one of an absorbent, a suitable porous material, a pore system, and a material with a rough or rugged surface. The main advantage of utilizing a pore structure of a material is that the material can be designed to exhibit hygroscopic properties, due to the fact that there is a significant relationship between the geometric structure of the pore system and the their ability to absorb water and moisture. Further, a porous system can also be monitored for temperature.

In another preferred embodiment of the present invention the pore system is continuous.

In a preferred embodiment of the invention the sensor comprises an indicator and a carrier, where the carrier is an absorbent compound capable of absorbing water/water vapour from the surrounding air. According to this embodiment the indicator migrates through an absorbent in response to ambient humidity, i.e. when a predetermined relative humidity level is exceeded and the migration of the indicator ceases when the ambient relative humidity level is lowered below said predetermined level and also the migration is resumed when the relative humidity level again exceeds said predetermined level.

In a preferred embodiment of the invention the carrier is provided with an absorbent.

In another preferred embodiment of the invention the absorbent is a salt, preferably a hygroscopic salt. Here the invention takes advantage of the relative humidity equilibrium of a saturated salt solution. A hygroscopic salt stands in equilibrium with the moisture content of the surrounding air. For many salts, the equilibrium follows lows the relative humidity. If the ambient air contains more moisture than the equilibrium relative humidity of the salt, the salt will absorb water and alternatively, a humid salt will give away moisture at equilibrium relative humidity. This phenomena is used to design a irreversible sensor or a irreversible moisture indicator.

Further, this phenomena is utilized so that the salt dissolves into free water, and thereby give rise to a colour change, or that the salt allows to be absorbed into an absorbent according to the present invention.

In a more preferred embodiment of the invention the salt is at least one of, or a combination of, NaCl, KCl, LiCl, LiBr, ZnBr₂, KOH, LiI, K₂C₂R₃OH, MgCl₂, NaI, K₂CO₃, Mg(NO₃)₂, NaBr, Kl, NaNO₃, KBr, (NH₄) ₂SO4, K₂NO₃ and K₂SO4. As hygroscopic active substance, inorganic salts are preferable since they have a defined equilibrium relative humidity.

In a preferred embodiment of the invention the hygroscopic material is at least one of a hygroscopic thickening agent, a consistency agent and a gelation agent. Preferable, these agents are at least one of (or any combination thereof) E400-E495 and E1404-E1451 (see table 1). The use of a hygroscopic thickening agent or a hygroscopic composition as a carrier enables many new applications of the sensor according to the invention. One advantage using a hygroscopic thickening agent, composition or mixture is that the carrier directly can be printed on, sprayed on, painted on and/or coated on a surface of an item. Preferable, the mentioned surface is modified, for example by hydrophobing, before printing on, spraying, painting and/or coating the hygroscopic thickening agent, (composition or mixture) onto the surface of an item in order to keep the accuracy of the sensor. Herein the term item includes almost everything having a surface being exposed to a surrounding, for instance a pill, a tablet, a paper, a plastic material, cellulose, a fibre, a label, a packing, a wrapping, a board and the like. The item is exposed to a humidity at least one of the processes of manufacturing, printing, packing, wrapping, storing, transportation and handling.

In a more preferred embodiment the hygroscopic thickening agent is one of a gel compound, a gel matrix, gelatine and potato flour.

Further, the gel matrix comprises at least one of a hygroscopic component and an indicator. Other components that may be included in the gel or gel matrix, in order to prevent transparency of the gel, is for example TiO₂ which will be permeable for the indicator.

In a preferred embodiment of the invention the carrier further comprises a hygroscopic thickening agent for keeping water in the carrier. This is particularly important for sensors indicating a cumulative time period of presence of an ambient relative humidity at lower levels of relative humidity (RH).

In a preferred embodiment of the present invention asuitable porous material has a continuous pore system and is at least one of a fibre, paper, wood, corrugated fibreboard, woven or non-woven materials and clay.

In a preferred embodiment of the invention the indicator is a colour indicator such as dye, pigment, hue and paint. A colour shift according to the invention is indicated in different ways.

In a preferred embodiment of the invention the carrier is arranged to allow migration of the indicator from the interior of the carrier to its surface, allowing visual detection of a user.

In another preferred embodiment of the invention the carrier is arranged to allow migration of the indicator at a surface layer of the carrier, also allowing visual detection of a user. It is possible for a sensor according to the present invention to at least change colour or use another optical property as an indicator, for example a dye, migrating within a carrier of the sensor or along an observed surface layer of the carrier. Preferably, the indicator, such as a dye, is water-soluble or partly water-soluble. Some indicators requires free water to be able to migrate. The indicator is preferable designed in order to prevent migration within a hygroscopic carrier and/or free salt-containing water. It is not necessary for the indicator to react chemically with for instance specific salts or the surface of a carrier. A clearer shift is obtained by utilising chemical or physical bonding to the carrier or to an absorbent component. Further, suitable indicators may be chosen from ions, molecules, and polar organic molecules of a certain size and the indicator can be in the form of a solution, suspension or emulsion.

In a preferred embodiment of the invention the indicator is a dye used in the food industry. One advantage using dyes for the food industry is that most of these dyes are non-toxic. Preferably, at least one of or any combination of E100 cur cumin, E101 riboflavin, riboflavin-5′-phosphate, E102 tartrazine EI04, quinoline yellow, E107 yellow 7G, E110 sunset yellow FCF, orange yellow S FD&C Yellow No.6, E120 cochineal, carminic acid, carmines, E122 azorubine, carmoisine, E123 amaranth FD&C Red No.2, E124 ponceau 4R, cochineal Red A FD&C Red No.4, E127 erythrosine FD&C Red No.3, E128 red 2G, E129 allura red AC FD&C Red No.40, E131 patent blue V, E132 indigotine, indigo carmine FD&C Blue No.2, E133 brilliant blue FCF FD&C Blue Dye No.1, E140 chlorophylis, chlorophyllins, E141 copper complexes of chloropyll and chlorophyllins, E142 green S, E150(a) plain caramel dark brown colour made from sucrose, E150(b) caustic sulphite caramel see E150(a), E150(c) ammonia caramel see E150(a), E150(d) sulphite ammonia caramel see E150(a), E151 brilliant black BN, Black PN, E153 vegetable carbon, E154 brown FK, E155 brown HT (chocolate), EI60(a) carotene, alpha-, beta-, gamma-, E160(b) annatto (arnatto, annato), bixin, norbixin, EI60(c) paprika extract, capsanthin, capsorubin, EI60(d) lycopene, EI60(e) beta-apo-8′-carotenal (C 30), EI60(f) ethyl ester of beta-apo-8′-carotenic acid (C 30), E161(b) xanthophylls-lutein, E161(g) xanthophylls-canthaxanthin, E162 beetroot red, betanin, E163 anthocyanins, E170 calcium carbonate, E171 titanium dioxide, E172 iron oxides and hydroxides, E173 aluminium, E174 silver, E175 gold, E180 latolrubine Bk and E181 tannic acid and tannins is used. Similar compounds or analogues to the E-dyes above, sharing the same properties, may also be used as a indicator dye in the present invention.

In another preferred embodiment of the invention the sensor is designed in such a way that the importance of the durability of the colour indicator is decreased. In such an embodiment, the sensor comprises at least one carrier and an indicator, wherein the colour of the indicator is visible (detectable) before the sensor is exposed to the ambient surroundings. If the ambient relative humidity level exceeds a predetermined level the indicator starts to migrate (or diffuse) from one or more limited portions of the carrier. If the colour of the indicator disappears, the predetermined relative humidity level is widely exceeded, and if the colour of the indicator is still there (visible) the sensor is unexposed. One advantage with this type of sensor is that the sensor has a much longer length of life, even if the dye isn't stable in a salt or in an ambient environment.

In another embodiment of the invention the extension by migration of an indicator is related to a colour shift of a non-migrating indicator located at one or more limited portions on the carrier. Preferably, the sensor allows transport or migration of e.g. a pH-alteration within or at a surface layer of the sensor, wherein a non-migrating indicator changes colour when the pH-value is varied.

A further object of the invention is to achieve a method for reading a cumulative time period of presence of an ambient relative humidity indicated by a sensor and to achieve a method for providing a sensor carrier with an indicator.

In a preferred embodiment of the invention a method for reading the cumulative time period of presence of an ambient relative humidity indicated by a sensor mentioned before comprises the steps of; a) observing the extension of the indicator having extended by migrating from one or more limited portions, when the ambient relative humidity level have exceeded a predetermined level, and b) establishing the cumulative time period of presence of an ambient relative humidity by relating the extension of the indicator to a time period reference, is used. One advantage by using this method is that it enables an indication of for how long the ambient relative humidity level has exceeded a predetermined humidity level. From that information a user is able to estimate the damage of the item being exposed to an ambient surrounding.

In another preferred method of the invention a time period reference is used. Preferably, the time period reference is at least one of arranged in relation to the carrier, a stand alone tool for physical alignment to the extension of the carrier at the time of the measuring of the extension and a table comprising relations between extension and time. It is desirable to aim at and design a user-friendly time period reference.

In a preferred method of the present invention an external light source is used to assist in the visual detection. Preferably, the light source is one of a infra red light source, an ultra violet light source and a visible monochromatic light source.

Further, the detection of the sensor can be a colour shift directly visible to the eye or a “hidden” colour shift, which may be visible under a source of for instance ultraviolet- or infrared light. It is also possible that the indicator can be developed using chemical or physical reactions in able to detect a shift. The indicator may also give rise to changed surface properties of the carrier, which preferable will be visible for the naked eye.

In a preferred embodiment of the invention a method for providing a sensor for indicating a cumulative time period of presence of an ambient relative humidity is used by providing a carrier with an indicator, wherein the indicator is arranged at one or more limited portions of the carrier. This gives a good flexibility. Preferably, the indicator may be arranged within the carrier and/or directly on the surface layer of the carrier, for instance on the bottom and/or the upper side of the carrier.

In another preferred method of the present invention the carrier is a thickening agent mentioned before and the method further comprises the step of painting, spraying, printing on or partial coating of a surface of an item. This method opens up for many new commercially opportunities. Preferably, the carrier is a thickening agent (or sensor composition) being arranged to a surface of an item before the indicator is arranged to one or more limited portions of the thickening agent (or sensor composition).

In a preferred embodiment of the invention the thickening agent is sprayed direct on a pharmaceutical and/or nutritional additive, preferably in the form of a pill and/or a tablet.

In another preferred embodiment of the invention the thickening agent is printed on at least one of a paper, a fibre, a card and/or a label. Further, the thickening agent is preferably partial coating of a surface of packaging.

In more preferred embodiment according to the invention the method further comprises the step of pretreating, modifying, preferably hydrophobing the actual surface of an item. This step facilitates the migration of the indicator within or at a surface layer of the thickening agent and not within the actual item.

In a preferred embodiment of the invention a method for providing a sensor for indicating a cumulative time period of presence of an ambient relative humidity by providing a carrier with an indicator a carrier according to the invention is dipped into a salt solution. Alternatively, the salt solution being sprayed on to the carrier. One advantage with this methods is that they enables to get enough absorbent, salt solution, into the carrier.

In another preferred embodiment of the invention the salt solution is pre-heated. Further, the carrier includes an absorbent component subsequently being dried in warm air. After drying, the indicator is preferable applied on one side of the carrier.

In one embodiment of the invention the carrier is further being wind upon a roll. Alternatively, polyeten or a double adhesive tape is rolled in the roll at the same time. Thereby, problems with indicators being transferred to the upper side and/or to the lower side of the carrier is avoided. It is also possible that a doped carrier, or another of the above-described carriers, may be adhesively joined with an adhesive layer permitting water transport through at least one layer of the carrier. This is achieved either by designing the adhesive layer so that it in itself permits the moisture transport, or by making the space in between open by not covering the whole surface between the dye carrier and the salt carrier. The advantage of dividing the dye and carrier is that the handling of the materials is simplified. The manufacture of the present sensor also gets more flexible as it lowers the storage and less complicated equipment may be used for the manufacture.

Further, the sensor can be provided with a protection cover, preferably of a liquid impervious material. The cover being pulled of during activation of the sensor. Thereby, the cover has an improved protection until it has been pulled off.

In a preferred embodiment of the present invention the method further comprises the step of adding a water keeping compound to the carrier. On advantage using a water keeping compound is that the agitation of gravitation, specially on sensors indicating at lower relative humidity, is decreased.

In a preferred embodiment of the invention a sensor for indicating a cumulative time period of presence of an ambient relative humidity, comprises an indicator and a carrier and the indicator is located at one or more limited portions of the carrier, the carrier allowing migration of the indicator within the carrier, wherein the indicator causing an extension thereof by migrating from the one or more limited portions when the ambient relative humidity level exceeds a predetermined level. The extension by migration is detectable by a user and the cumulative time period is related to the extension by migration and the characteristics of the indicator and the carrier. The sensor according to the present invention is for use in at least one of the processes of manufacturing, printing, packing, wrapping, storing, transportation and handling of an item, namely, processes in which an item is, or may be, exposed to the ambient surroundings.

In a preferred embodiment of the invention the sensor is at least one of a sensitive items humidity sensor, a food stuff humidity sensor, a building- or construction material humidity sensor, a pharmaceutical humidity sensor, an electronic equipment humidity incorporating at least one of the characteristics of the sensor described above and prepared according to at least one of the previously mentioned processes. One large advantage with the sensor according to the present invention is that the sensor may be used in a very wide range of different business areas. The sensor is easily used everywhere where there is a risk that a certain item is exposed to moisture. For instance, it is of interest to indicate the amount of moisture in boats, vehicles, buildings and/or houses. Also, military materials, such as weapons, are sensitive to moisture.

Further, it is of interest for suppliers of electronic equipment to indicate wherever the equipment, such as mobile phones, is exposed to humidity under transportation and/or use. For example, it is important for suppliers or sellers to prove that a user has dropped his/hers mobile phone into water or the like.

In a preferred embodiment of the invention the sensor is provided with an additional component, namely a leakage-preventing barrier arranged to cover the surface layer of the sensor. The purpose of using a barrier is to protect the surroundings from leakage and above all the items on which the sensor is applied. This also protect the sensor from being contamined from the surroundings. Further, the leakage-preventing barrier can be a membrane. In a preferred embodiment of the invention a leakage-preventing barrier is a semi-permeable membrane, permeable for water vapour and/or steam, but not permeable to liquid water, in one direction, i.e. into the sensor. In addition, the membrane may also be a protection against minor mechanical damages. For instance the membrane may be made of EPTFE (expanded polytetraflouroethylene, more known under the trademark Goretex).

In another preferred embodiment of the invention a sensor is sprayed directly on a actual item or at a packing including sensitive goods or objects. One or more sensors may also be arranged in a container during transportation or storing of items who being sensitive for humidity, such as vegetable food, fruit foods and/or other provisions.

### Definitions

As used herein the term thickening agent is interpreted to include a consistency agent and/or a gel agent.

As used herein especially used in claim 1 the term within, means that a indicator according to the invention, located at one or more limited portions of an carrier, is arranged to migrate in, at or on the carrier, preferably from the interior of the carrier to its surface and/or at a surface layer of the carrier.

### BRIEF DESCRIPTION OF THE DRAWINGS

The advantages and details of the present invention will become apparent from the following description when taken in conjugation with the accompanying drawings, in which;

FIG. 1a) shows one embodiment of a sensor according to one embodiment of the present invention and FIG. 1b) illustrates how a cumulative time period of presence of an ambient relative humidity being related to a predetermined humidity level and the extension by migrating of a indicator.

FIG. 2 shows a another embodiment of the sensor.

FIG. 3 shows a sensor exhibiting a circular shape having the migration of a indicator towards the edges.

FIG. 4 shows a sensor according to the invention provided with a time scale.

FIG. 5 shows multiple sensors arranged on the same goods for different intervals of relative humidity.

FIG. 6 shows an alterative embodiment of a sensor.

FIG. 7 shows yet another embodiment of a sensor.

### DETAILED DESCRIPTION OF PREFERRED EMBODIMENTS

A number of preferred embodiments of a sensor for indicating a cumulative time period of presence of an ambient relative humidity will be disclosed below.

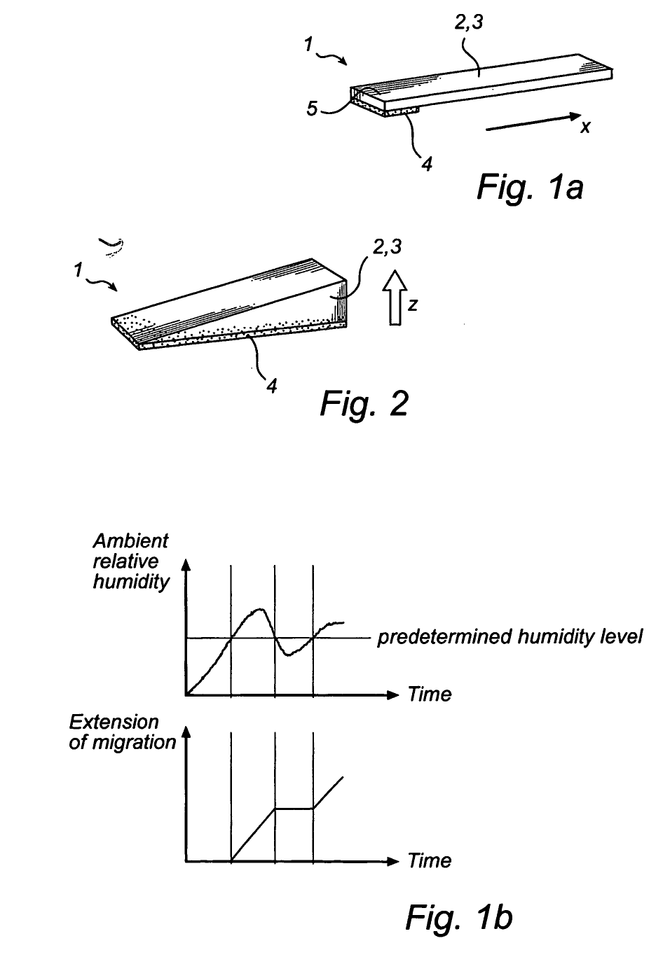

A first embodiment of a sensor for indicating a cumulative time period of presence of an ambient relative humidity is presented in FIG. 1a. The sensor 1 comprises a carrier 2 including an absorbent component 3 and an indicator 4 being located at a portion of the carrier. Here, the main purpose of the carrier 2 is to provide a support material to keep the components of the sensor assembled and to prevent convection or occurrence of other undesired transport mechanisms. The absorbent 3 is capable of absorbing water/water vapour from the surrounding air. The absorbent component 3 is designed to allow the indicator 4 to migrate through and/or within said carrier 2 including an absorbent component 3 in response to said ambient humidity and the indicator causing an extension by migrating from the one or more limited portions when the ambient relative humidity level exceeds a predetermined level. When the relative humidity level is lowered below said predetermined relative humidity level the migration of the indicator 4 ceases. The indicator 4 resumes migration when the ambient relative humidity again exceeds said predetermined level. This procedure is repeated until the indicator 4 has migrated through the whole carrier 2. FIG. 1b) illustrates the cumulative time period of presence of an ambient relative humidity level being related to the extension by migrating. In figure la it is assumed that the sensor 1 is arranged to (an item in question. Preferably, the sensor has a bottom portion comprising a fastening means to allow attachment to an item being exposed to the ambient surroundings. In the embodiment as shown in FIG. 1, the sensor 1 has an essentially rectangular shape exhibiting a longitudinal migration direction x. Before activation, the indicator 4 is located at a limited portion 5 of the carrier 2. Upon exposure of a predetermined level of ambient relative humidity, the indicator 4 migrates in the direction x of the absorbent component 3 along the sensor in response to said ambient humidity.

In a second embodiment of the invention the indicator 4 is arranged onto the bottom portion of a carrier with hygroscopic characteristics. The carrier 2 can exhibit a wedge-shaped cross-section, as shown in FIG. 2, exhibiting a vertical migration direction z. In this embodiment, the indicator 4 migrates within the carrier preferably in the z direction, wherein the z direction is defined to be approximately upwards through the carrier 2 in response to said ambient humidity. As the indicator 4 moves within the carrier 2 the exposure time can be estimated due to its wedge-shape, since it will take longer for the indicator 4 to migrate in one first end through the thicker layer of the carrier 2, than for the indicator 4 to migrate through the thinner layer of a second end. Further, it is also possible that the carrier includes an absorbent component.

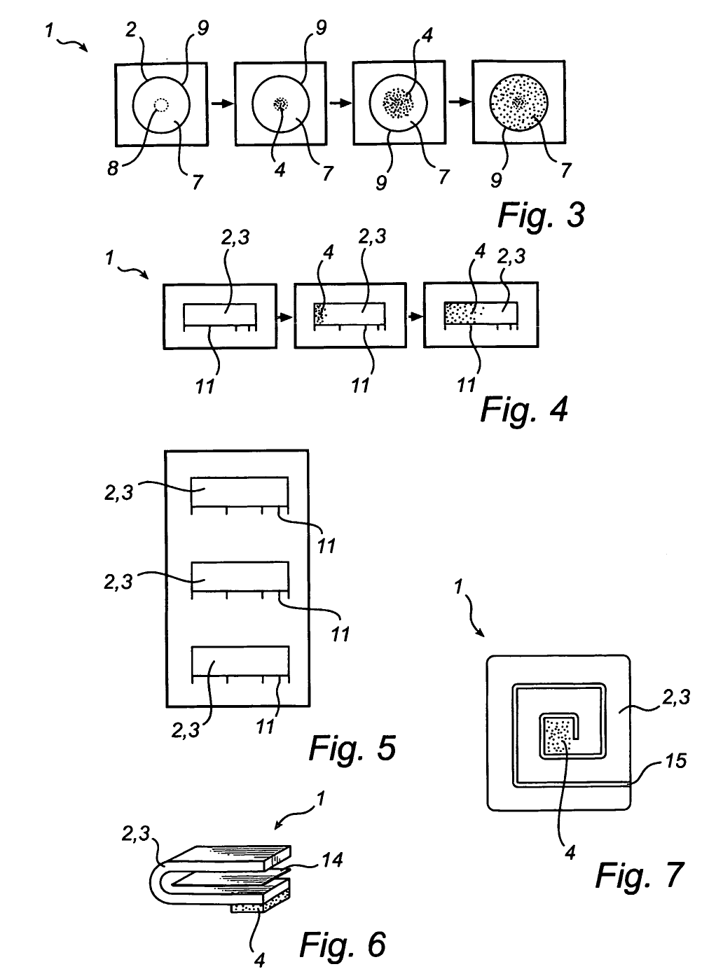

In a third embodiment of the invention shown in FIG. 3, the sensor 1 has an essentially circular shape carrier 2, including an absorbent component 3, exhibiting a surface layer area 7. Before activation, the indicator 4 is arranged preferably in the central part 8 of the carrier. The indicator causes an extension by migrating from the surface area 7 towards the edges 9 of the carrier, when the ambient relative humidity level exceeds a predetermined level. The extension by migrating is detectable by a user.

In a fourth embodiment of the invention the sensor comprising a carrier and an indicator for indicating of a cumulative time period of presence of an ambient relative humidity. The carrier here is a thickening agent, such as a gel matrix, and being painting on, spraying, printing, and partial coating of a surface of an item. Further, the indicator is arranged to one or more limited portions of the carrier. (Not shown.)

In a fifth embodiment, as shown in FIG. 4, the sensor may be provided with a time axis 11 in order to determine the exposure time for a certain level of relative humidity. The sensor may be designed with several absorbent components 3 arranged to each other having different relative humidity equilibrium properties, i.e., the time axis 11 will then indicate the respective exposure times for different levels of relative humidity as shown in FIG. 5.

The sensor according to the invention may be provided with a fourth component, namely a leakage-preventing barrier 10 arranged to cover preferably a outer surface of the sensor. The leakage-preventing barrier may be a membrane, such as a semi-permeable membrane.

The sensor may also exhibit a folded structure as shown in FIG. 6. In this embodiment the sensor is folded once and the indicator 4 is arranged in the bottom portion of the folded sensor. An impervious membrane 14 is arranged between the folded carrier portion 2, preferably including the absorbent component 3. In this way the shift of the indicator 4 is delayed and the size of the sensor is decreased.

In a sixth embodiment, the sensor exhibits another folded spiral-shaped structure as shown in FIG. 7. In this embodiment the sensor is folded having the indicator 4 arranged in the core portion of the folded sensor. An impervious membrane 15 is arranged on the wrapped carrier 2 including the absorbent component 3. In this way the shift of the indicator 4 is delayed and the size of the sensor may be decreased.

A sensor according to the invention can also be provided with a “window” on the sensor in order to monitor a certain relative humidity interval. In this way it is possible to use the same sensor for different applications.

In a seventh embodiment of the invention a method for reading the cumulative time period of presence of an ambient relative humidity indicated by a sensor according to the invention comprises the steps of; observing the extension of the indicator having extended by migration from one or more limited portions, when the ambient relative humidity level have exceeded a predetermined level; and establishing the cumulative time period of presence of an ambient relative humidity by relating the extension of the indicator to a time period reference. The time period reference is one of; arranged in relation to the carrier, a stand alone tool for physical alignment to the extension of the carrier at the time of the measuring of the extension and a table comprising relations between extension and time.

It should be understood that many modifications of the above embodiments of the invention are possible within the scope of the invention such as the latter is defined in the appended claims.

### Experiment

The invention will now be closer illustrated by means of the following non-limiting examples. These examples are set forth merely for illustrative purposes and many other variations of the method may be used.

### EXAMPLE 1

Preparation of Sensors

A glass fibre filter of the type MGA (Munktell) was used as carrier. The salt KCl (85% RH, quality p.a.) was used as absorbent component and quinoline yellow (BI04), caramine (E120) and patent blue (B131) (food quality) respectively, were used as indicator dyes. The deionised water used exhibited a quality of 18 M ohm/cm.

The carriers have been prepared in two different ways with the same results, either by dipping the carrier into a concentrated salt solution (KCl) or by spraying a salt solution on one side of the carrier. In this case the other side of the carrier was vented. The doped carrier is then dried to a stable weight. The indicator dye, which is dispersed in isopropanole, is applied on one side of the dried and doped carrier. The sensor is then placed in a climate chamber having a relative humidity (RH) level lower than 55% RH.

Treatment of Sensors in a Climate Chamber

The test samples of the different sensors had a size of about 2 cm². Further, there were three of each type and a reference sample. The test samples were placed in a pressure moisture chamber having an accuracy regarding the relative humidity level of about 0.5% RH and the desired levels of the relative humidity were set to the desired values. The test course was as follows: 75%, 75%, 80%, 83%, 86%, 89% and 50% RH. The measurements were performed on each level with a time period of at least 24 hours between the observations. All the measurements were made at the temperature of 20° C.

Changes in the Weight of the Sensors

The test samples were weighed on a balance a resolution of 0,001 g. The samples exhibited no weight change at 75%, 80% and 83% RH. At 86% RH, the test samples have increased their weight of about 0.05 g and another weight increase of 0.20 g. The initial weight of the test samples were resumed at 50% RH. The difference in the weight and the weight change between the samples was not significant and could therefore be neglected. The weight increase is assumed to be water being absorbed by the test samples, since it was the only parameter being changed during the test cycle.

Visual Observations

The test samples were visually observed in respect of their colour shift and migration distance. The indicator dyes starting to migrate at a certain RH value for all sensors having different indicator dyes applied on them. In the case of quinoline yellow (EI04) it occurred at a RH value exceeding the equilibrium for the salt in the carrier at 86% RH. Caramine (EI20) was somewhat a head in this test at 80% RF and in the case of patent blue (EI31) the shift occurred essentially before the estimated value, i.e., at the first level of the cycle, that is 75% RH.

The indicator migrates about 8-10 mm in the first 24 hours and then it will take the indicator about 72-130 hours to migrate 20 mm. This is true for all three indicator dyes. The front line of the migration of the indicator-dye is very clear and is easily seen by the naked eye.

As is evident from example 1, the EI04 without modifications turned out to be not good enough for use in a sensor according to the invention, whereas caramine and patent blue exhibited excellent properties, such as clear and significant shifts.

The example proves that the technology works by allowing transport of a water-soluble dye in a saturated salt solution, absorbed in the absorbent component of the sensor, as a response to ambient relative humidity levels. The test also proves that it is possible to accumulate the exposure to moisture during a period of time.

Combinations of different kinds of carriers, salts and indicator dyes will lead to the optimal sensor for the desired purpose regarding use, temperature and moisture content and falls in to the scope of the invention. Further, for the same reasons using a sensor comprising a carrier with hygroscopic properties and a carrier will allow migration of the indicator within the carrier when a ambient relative humidity level is exceeding a predetermined humidity level and an accumulative time period being related to the extension by migration and the characteristics of the indicator and the used carrier.

The invention shall therefore not be limited to the above shown embodiments and examples, but shall be interpreted within the scope of the appending claims.

TABLE 1
E400-E495

| | | |
|---|---|---|
| E | 400 | Alginic acid |
| E | 401 | Sodium alginate |
| E | 402 | Potasssium alginate |
| E | 403 | Ammonium alginate |
| E | 404 | Calcium alginate |
| E | 406 | Agar |
| E | 407 | Carrageenan |
| E | 407a | Processed Euchema Seaweed |
| E | 410 | Locust bean gum |
| E | 412 | Guar gum |
| E | 413 | Tragacanth |
| E | 414 | Acacia gum (Gum arabic) |
| E | 415 | Xanthan gum |
| E | 417 | Tara gum |
| E | 418 | Gellan gum |
| E | 422 | Glycerol |
| E | 440 | Pectins, Amidated pectin |
| E | 460 | Microcrystalline cellulose, Powdered cellulose |
| E | 461 | Methyl cellulose |
| E | 463 | Hydroxypropyl cellulose |
| E | 464 | Hydroxypropyl methyl cellulose |
| E | 465 | Ethyl methyl cellulose, Ethyl hydroxyethyl cellulose |
| E | 466 | Carboxy methyl cellulose, Sodium carboxy methyl cellulose |
| E | 469 | Enzymatically hydrolysed carboxymethylcellulose |
| E | 470a | Sodium, potassium and calcium salts of fatty acids |
| E | 470b | Magnesium salts of fatty acids |
| E | 471 | Mono- and diglycerides of fatty acids |
| E | 472a | Acetic acid esters of mono- and diglycerides of fatty acids |
| E | 472b | Lactic acid esters of mono- and diglycerides of fatty acids |
| E | 472c | Citric acid esters of mono- and diglycerides of fatty acids |
| E | 472d | Tartaric acid esters of mono- and diglycerides of fatty acids |
| E | 472e | Mono- and diacetyl tartaric acid esters of mono- and diglyc- |
| | | erides of fatty acids |
| E | 472f | Mixed acetic and tartaric acid esters of mono- and diglyc- |
| | | erides |

E1404-E1451

| | | |
|---|---|---|
| E | 1404 | Oxidized starch |
| E | 1410 | Monostarch phosphate |
| E | 1412 | Distarch phosphate |
| E | 1413 | Phosphated distarch phosphate |
| E | 1414 | Acetylated distarch phosphate |
| E | 1420 | Acetylated starch |
| E | 1422 | Acetylated distarch adipate |
| E | 1440 | Hydroxy propyl starch |
| E | 1442 | Hydroxy propyl distarch phosphate |
| E | 1450 | Starch sodium octenyl succinate |
| E | 1451 | Acetylated oxidised starch |

1. Sensor for indicating a cumulative time period of presence of an ambient relative humidity, the sensor comprising an indicator and a carrier, the indicator and the carrier being humid at approximately the same time, the indicator being located at one or more limited portions of the carrier;
the carrier allowing migration of the indicator within the carrier;
the indicator causing an extension thereof by migrating from the one or more limited portions when the ambient relative humidity level exceeds a predetermined level;
the extension by migrating being detectable by a user; and
the cumulative time period being related to the extension by migration and the characteristics of the indicator and the carrier.

2. Sensor according to claim 1, wherein the carrier is a hygroscopic material.

3. Sensor according to claim 2, wherein the hygroscopic material is one of an absorbent and a suitable porous material.

4. Sensor according to claim 2, wherein the hygroscopic material is a hygroscopic thickening agent.

5. Sensor according to claim 4, wherein the hygroscopic thickening agent is one of a gel compound, a gel matrix and gelatine.

6. Sensor according to claim 3, wherein the absorbent is a salt.

7. Sensor according to claim 1, wherein the carrier further comprises a hygroscopic thickening agent for keeping water in the carrier.

8. Sensor according to claim 3, wherein the suitable porous material has a continues pore system and is one of fibre, paper, wood, corrugated fibreboard, woven materials and clay.

9. Sensor according to claim 1, wherein the indicator is a colour indicator such as dye, pigment, hue and paint.

10. Sensor according to claim 9, wherein
the colour of the indicator is visible to a user before use of the sensor; and
the colour of the indicator still is visible until a predetermined cumulative time period of presence of an ambient relative humidity is exceeded.

11. Sensor according to claim 1, wherein the carrier is arranged to allow migration of the indicator from the interior of the carrier to its surface, allowing visual detection of a user.

12. Sensor according to claim 1, wherein the carrier is arranged to allow migration of the indicator at a surface layer of the carrier, allowing visual detection of a user.

13. Sensor according to claim 1, wherein the extension by migration of the indicator is related to a colour shift of a non-migrating indicator located at one or more limited portions on the carrier.

14. Method for reading the cumulative time period of presence of an ambient relative humidity indicated by a sensor according to claim 1, comprising the steps of:
observing the extension of the indicator having extended by migrating from one or more limited portions when the ambient relative humidity level have exceeded a predetermined level; and
establishing the cumulative time period of presence of an ambient relative humidity by relating the extension of the indicator to a time period reference.

15. Method according to claim 14, wherein the time period reference is one of
arranged in relation to the carrier;
a stand alone tool for physical alignment to the extension of the carrier at the time of the measuring of the extension; and
a table comprising relations between extension and time.

16. Method according to claim 14, wherein an external light source is used to assist in the visual detection.

17. Method according to claim 16, wherein the light source is one of an infra-red light source, an ultra violet light source and a visible monochrome light source.

18. Method for providing a sensor for indicating a cumulative time period of presence of an ambient relative humidity by providing a carrier with an indicator, the indicator being arranged to one or more limited portions of the carrier, the indicator and the carrier being humid at approximately the same time.

19. Method according to claim 18, wherein the carrier is a thickening agent and further comprises the step of painting, spraying, printing on, or partial coating of a surface of an item.

20. Method according to claim 19, further comprising the step of hydrophobing the surface.

21. Method according to claim 18, wherein the carrier is dipped into a salt solution.

22. Method according to claim 18, wherein a salt solution is sprayed on the carrier.

23. Method according to at least one of claim 18 and 19, further comprising the step of adding a water keeping compound to the carrier.

24. A sensor according to claim 1 for use in the processes of manufacturing, printing, packing, wrapping, storing, transportation and handling.

25. Sensitive items humidity sensor incorporating the sensor according to claim 1.

26. Food stuff humidity sensor incorporating the sensor according to claim 1.

27. Building material humidity sensor incorporating the sensor according to claim 1.

28. Pharmaceutical humidity sensor incorporating the sensor according to claim 1.

29. Electronic equipment humidity sensor incorporating the sensor according to claim 1.
